SEQUENCE LISTING (8 sequences)

SEQ ID NO: 1 - DNA, 5278 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgatggctagccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 2 - DNA, 5278 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgacggctagccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 3 - DNA, 5337 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgatggctagccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 4 - DNA, 5337 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgacggctagccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 5 - DNA, 5358 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgatggctagcaagtccaacaagcaagtaactcctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 6 - DNA, 5358 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgacggctagcaagtccaacaagcaagtaactcctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 7 - DNA, 5412 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagctatggtgcatggcaacactctttcatatgcagttcgcacccaggacgggatgtcgggtgcaccagtctgtgacaaatatggtcgggtgttagcagtccatcaaacaaacactgggtacactggaggtgctgtcataatagacccagcagacttccatccagtgaaagccccatctcaggtggaattgctcaaagaggaaatagagcggctaaaagctcaactgaactctgccactgagaacgcaacgactgtagttacacaacaacctagtgctgcactagaacagaaaagtgtcagcgatagtgatgtagttgaccttgtcagaactgcaatggaacgtgagatgaaggtgctgcgtgatgaaatcaatggaatacttgcaccattcctacaaaaaaagaaaggtaagaccaagcatggtaggggtagagtcaggcgtaaccttagaaaaggtgtgaaacttcttaccgaggaagagtatcgagaactcttagagaaaggtcttgatcgtgagacattccttgatctcatagaccgcattattggtgagaggtctggctaccctgactatgatgatgaagattactatgatgaagatgatgatggctggggaatggttggtgatgatgtagaatttgattatactgaagtaattaactttgaccaagcaaaaccaattcctgccccgagaacaaccaagcaaaaaatttgccccgaaccagaagtcgaatcacaaccacttgatttgtcccaaaagaaagaaaaacaatcagaatatgaacaacaagtggtgaagtctaccaagcctcaacaattagaacatgaacaacaagtggtgaagcctatcaagcctcagaagagtgagcctcaaccatactcacaaacttacggcaaggcaccaatctgggaatcttacgattttgactgggatgaggatgatgccaagtttattctgccagcgccacaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgatggctagcaagtccaacaagcaagtaactgaaggccggggcagtctgctgacgtgcggcgacgtagaagaaaatcctggtccccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

SEQ ID NO: 8 - DNA, 5388 bp, Human astrovirus
ccaagaggggggtggtgattggcctttggcttatcagtgtatagataacatttctctgaccgtttaccacacaattaacaacaatatggcatacggtgagccatactatagctctaaacctgacaaagatttcaattttggaagcacaatggcacgtaggcagatgacacctactatggtaacaaagcttcccaaatttgttaggaattctccacaagcttatgattggatcgtaagaggtctgatctttcccaccattggtaaaacttatttccaacgagttgttgtgattactggtgggcttgaggatggaacatatggctcattcgcatttgacggtaaagagtgggtagggatctacccaatagagcacttaaatctcatgtcatctttgaaactgatacacaaagccaacgctcttcaggagagactgcgtctctcccaagaagagaaagccaccctcgctctggatgtgcaattccttcaacatgaaaacgtgcgattgaaggagatgatcccaaagccagagccacggaaaatacagatgaagtggataataatgggagcagtgcttacattcttatctctgatacctgggggttatgcgcacagccagaccaacaacaccatatttactgacatgatagctgcctgcaagtactcaactgagacactaacagaaaatcttgaccttagaatcaagcttgcactagcaaacataaccattagtgataagctagatgctgtgaggcaaattcttaactttgcctttgtgcccagagcccattggttgagaactgtgttctattatatccattactatgaaatgtggaatatttttatgtttgttcttgctattggcactgtcatgaggagcgcccgccctggtacagacttggttacacttgcaacatcccacttgtctggttttaggatggctgtcctacccacaattccatttcacaccactatgactttgtgggttatgaacacacttatggtttgttattattttgacaacttgctagcaataacattggcaatcttagcaccaattcttggcattatcttcttgtgcttcatggaagactccaactatgtgagccagatacgtggccttattgctacagcagtattaattgctggtgggcatgcatgtttgacactcacaggcacgaccacgtcattgtttgttgtcatactaacctgtaggttcatacgtatggcaactgttttcattggcaccaggttcgagatccgtgacgctaatggaaaggttgtggccactgtaccaactaggattaaaaatgttgcatttgacttttttcagaagctgaagcagtcaggggtgcgagttggagtcaacgaattcgttgtcataaaaccaggtgcattatgtgttatagacacccctgaaggaaaagggacaggtttcttttctggcaatgacatagtaacagcagcacatgttgttggcaataatacttttgtgaatgtgtgctatgagggtttgatgtatgaagcgaaggtgcggtacatgcccgaaaaggatatagcattcttaacttgtcctggtgacctgcatccaacagcaagattaaaattatcaaagaacccagattatagttgtgtcacagttatggcttatgtgaatgaggatcttgtggtttcaaccgcagcagccatggtacatggcaacactctctcatatgcagttcgcactcaagacggaatgtcaggtgcaccagtttgtgacaaatatggtcgagtgttagcagtccatcagactaatactgggtacactggaggtgctgtcataatagacccagcagacttccatccagtgaaggccccatctcaggtggaattgctcaaagaggaaatagagcgattaaaagcccaattaaattccaccgctgagaatccagcgactgttgttacacaacaacctattgctacactagagcagaaaagtgtcagcgatagcgatgtgattgaccttgttagaactgcaatggaacgtgagatgaaggtgctgcgcgatgaaatcaatgggatacttgcaccgttcctacaaaaaaagaaaggtaagaccaagcatggtaggggtagagtcagacgaaaccttaggaaaggtgtgaaacttctcactgaggaagaatatcgagagctcttagagaaaggtctggatcgtgagacattcctagatctcatagaccgtattattggtgagaggtctggctaccctgactatgatgatgaggattattatgatgaagatgatgatggatggggtatggttggtgatgatgtagaatttgattataccgaagtaatcaattttgaccaagcaaaaccaactcctgccccgagaacaagtaagccacaacaagccaacacttctcaaaaaccccgccccgagctagaagctgaagcacaaccgcttgatttgtctcagaagaaagagaaacaaccagaacatgagcaacaagtggcgaagcctaccaagatgcagaagaatgaacctcaaccatattcacaaacttatggcaaggcaccaatctgggaatcctatgactttgattgggatgaggatgacgccaagttcattcttccagcgcctcaccggttgactaaggcagatgaaatagtccttggatccaaaatcgtcaagcttagaacgattattgaaacagccataaagactcagaattatagtgcattacctgaagcagtatttgagctcgacaaagcagcttatgaagcaggtttggaaggttttctccaaagggttaaatcgaaaaacaaggccccaaaaaactacaaagggccccagaagaccaaggggcccaaaactaccactcattagatgcatggaaattgttgctagagcctccgcgggagcgaaggtgcgtgcctgcgaattttccactattaggccatttaccaattaatagacccatctttgatgataagaaacccagggacgatctccttggcctacttccagaaccaacctggcatgctttcgaggaatacggaccaaccacatggggcccacaagcttttatcaaatcttttgataaatttttttatgcagaaccaattgactttttctcagaatatccacagttgtgtgctttcgctgattgggcaacttatcgcgagtttcggtatctagaggatactagagtgatacacataactgcaactgagaaaaacactgattcaacacctgcttatcctaaaatgaattattttgacactgaagaaaattacctggaagcacatgggtgggcaccatatattagagaattcactagggtctacaaaggagacaaacctgaagtactttggtacctatttcttaagaaagagatcattaaggaggaaaaaattaggaattctgatatccggcagatagtgtgtgccgaccccatttacaccaggataggggcgtgcttagaagcgcatcagaatgccttaatgaaacagcataccgacacctcagttggtcaatgtgggtggtcaccaatggaaggcggctttaaaaaaaccatgcagcgcctggtaaataaagggaataaacacttcattgagttcgactggacccgctatgatggaactataccaccagcactctttaaacacatcaaagaaattaggtggaatttcatcaataaagaccaacgtgaaaagtacagacatgtacatgaatggtatgttaacaacctccttaatcgccatgtacttctaccatctggtgaagtcaccttgcagacgcgaggtaatccatctggtcagttttcaacaacaatggataacaacatggttaacttttggttacaggcttttgagtttgcttattttaatggaccagacagagacctttggaagacctatgacactgtagtttatggagatgacaggctttctacaacaccttcggtgcccgatgattatgaggagagagtgatcactatgtatagagacatctttggcatgtgggttaagcctgggaaggttatctgtagagacagcatagttggattgtccttttgtggcttcactgttaatgaaaaccttgagcctgtgccaacttctcctgaaaagttaatggcatcactgttaaaaccttacaaaatattacctgatcttgaatcactccatgggaaactcctatgctatcagttgcttgctgcgttcatggcagaggaccacccctttaaggtgtatgtggagcactgcctctcgcggactgcaaagcagcttcgtgactctggccttccagccagactcacagaagagcaactccatcgcatttggaggggaggaccaaagaagtgtgatggctagcaagtccaacaagcaagtaactgaaggccggggcagtctgctgacgtgcggcgacgtagaagaaaatcctggtccccctaggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagacgcgtgcaaagcagcaggggaaatcaatccctgcacatctggaagccgcggccacgccgagtaggaacgagggtacagcttccttcttttctgtctctgtttagattattttaatcaccatttaaaattgatttaatcagaagcaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccacccgcgctgctaacaaagcccgaaaggaagctgagttggctgctgccaccgctgagcaataactagcataaccccttggggcctctaaacgggtcttgaggggttttttgctgaaaggaggaactatatgcgctcata

(19) United States
(12) Patent Application Publication
(10) Pub. No.: US 2022/0257747 A1
(43) Pub. Date: Aug. 18, 2022

(54) METHODS AND COMPOSITIONS OF ASTROVIRUS REPLICONS

(71) Applicant: Infectious Disease Research Institute, Seattle, WA (US)

(72) Inventor: Jesse ERASMUS, Lynnwood, WA (US)

(21) Appl. No.: 17/612,560

(22) PCT Filed: Jun. 10, 2020
(86) PCT No.: PCT/US2020/037094

§ 371 Date: Nov. 18, 2021

Related U.S. Application Data
(60) Provisional application No. 62/859,683, filed on Jun. 10, 2019.

(51) Int. Cl. A61K 39/12; C07K 14/005; A61P 37/04; A61P 31/12

(52) U.S. Cl. CPC A61K 39/12; C07K 14/005; A61K 2039/6018; A61P 31/12; A61P 37/04

(57) ABSTRACT

The present invention provides recombinant replicons and methods of their use for expression of a secreted protein of interest so as to induce an enhanced immune response.

The present invention relates to recombinant replicons for eliciting improved immune response and methods for using the same.

### BACKGROUND

A fundamental aspect of the use of vaccines to confer immunity is the delivery of an immunogenic agent to a subject so as to elicit a response from the subject's immune system. In particular, effective stimulation of the subject's adaptive immune system is important in developing long-term immunity to a particular pathogen. One approach for immunization against viral pathogens involves introducing independently replicable viral genetic material, i.e. a replicon, into host cells, leading to expression and secretion of antigenic proteins that stimulate the immune system.

Current replicon technology was originally conceptualized and developed based on the ability to package RNA replicons into virus-derived nucleocapsids with or without envelope glycoproteins, termed viral replicon particles (VRPs). This approach was necessarily restricted to larger viruses with adequate packaging capability that would allow for incorporation of large foreign genomic information. To satisfy this requirement, two commonly used VRPs are derived from alphaviruses or flaviviruses, which possess relatively large genomes (>11 kilobases) and particle diameter (>60 nm).

The advent of non-viral delivery of RNA utilizing various formulations that can protect against RNA degradation, has made it possible to utilize positive strand RNA viruses, which contain infectious RNA genomes, in developing naked-RNA replicons. Eliminating the need to package replicons within viral particles also eliminates the requirement to use replicons derived from larger viruses. However, this technology has remained focused on the use of alphavirus and flavivirus replicons, most likely due to their extensive historical use. Furthermore, alphavirus-based naked RNA replicons have proven to be efficacious as vaccine platforms due to their robust antigen expression kinetics, owing to their use of a subgenome encoding the heterologous gene of interest, as well as a robust induction of, and resistance to, the IFN-mediated antiviral state of the host. However, as decreasing the effective dose of replicon material is desirable in order to best avoid potential toxicity issues, genome size remains a consideration. Additionally, nucleic acid manipulation using recombinant DNA techniques is greatly simplified when working with smaller constructs, resulting in greater genetic tractability and more rapid development of vaccine candidates. Therefore, developing replicons with smaller genomes that exhibit the same or similar beneficial characteristics as existing platforms remains a desirable goal.

The present disclosure overcomes the shortcomings in the art by providing recombinant astrovirus replicons that fit this desired profile and are effective in inducing increased immune responses.

### SUMMARY

In accordance with the description, embodiments include a recombinant replicon nucleic acid comprising: a first open reading frame comprising a.) a subgenomic nucleic acid sequence encoding a protein of interest that can be secreted by a cell; b.) a second open reading frame comprising a nucleic acid sequence encoding a first astrovirus nonstructural protein (nsP1a) and including a hypervariable region; and c.) a third open reading frame comprising a nucleic acid sequence encoding a second astrovirus nonstructural protein (nsP1b) and including a subgenomic promoter that is situated so as to initiate transcription of the subgenomic nucleic acid sequence. In some embodiments, the recombinant replicon nucleic acid has the structure: c.→b.→a. In some embodiments, the recombinant replicon nucleic acid has a 7-methylguanylate cap at its 5′ end.

In some embodiments, the first open reading frame further comprises a subset of a nucleic acid sequence encoding an astrovirus structural protein (VP90). In particular embodiments, the subset consists of between 5 and 50 nucleotides. In other particular embodiments, the subset consists of 30 nucleotides.

In some embodiments, the recombinant replicon nucleic acid further comprises an astrovirus conserved sequence element beginning within the first open reading frame and extending beyond the 3′ end of the first open reading frame.

In some embodiments, the hypervariable region has an astrovirus genotype that is different from an astrovirus genotype of the recombinant replicon nucleic acid. In some of these embodiments, the astrovirus genotype of the recombinant replicon nucleic acid is HAstV VII and the astrovirus genotype of the hypervariable region is HAstV IV.

In some embodiments, the third open reading frame includes a translational upstream ribosome binding site.

In some embodiments, the first open reading frame further comprises a nucleic acid sequence encoding a peptide with ribosomal skipping properties. In particular embodiments, the peptide is a 2A peptide from Thosea asigna virus capsid protein (T2A).

Other embodiments include a nanoparticle comprising any of the above recombinant replicon nucleic acids. In some of these embodiments, the nanoparticle consists essentially of a nanostructured lipid carrier containing the recombinant replicon nucleic acid. Other embodiments include a formulation comprising a plurality of the nanoparticles in a pharmaceutically acceptable carrier.

In some embodiments, a method of treating a subject to confer an immunity on the subject comprises administering the above formulation to the subject and thereby eliciting an immune response in the subject. In some of these embodiments, the immune response includes CD4+ T cell activation.

In some embodiments, a composition comprises any of the above recombinant replicon nucleic acids in a pharmaceutically acceptable carrier.

In some embodiments an isolated cell comprises any of the above recombinant replicon nucleic acids. In other embodiments, a method of delivering a therapeutic amount of a protein of interest to a subject comprises administering an effective amount of the isolated cell to a subject, wherein the protein of interest is a therapeutic protein and the cells secrete and thereby deliver a therapeutic amount of the protein of interest to the subject.

In some embodiments, a method of secreting a protein of interest from a cell comprises introducing any of the above recombinant replicon nucleic acids into the cell under conditions whereby the protein of interest is secreted, and where the cell is in a cell culture thereby secreting the protein from the cell. Some embodiments further comprise the step of harvesting the protein of interest from the cell culture. Another embodiment includes a composition comprising the protein of interest produced from this method. In still other embodiments, a method of delivering a therapeutic protein of interest to a subject comprises administering the composition to the subject.

### BRIEF DESCRIPTION OF THE DRAWINGS

FIGS. 1A-C show the organization of a human astrovirus genome and the significance thereof for expressing a protein of interest. FIG. 1A shows a schematic of the genomic organization of Human Astrovirus (HAstV) including three open reading frames (ORFs), ORF 1a, ORF 1b, and ORF 2, and also shows the location of highly conserved sequence elements flanking ORF 2 that are proposed to play a role in subgenome transcription and translation. FIG. 1B shows a table depicting four replicons of HAstV encoding NanoLuc® luciferase (nLUC) and each possessing one of four combinations of modifications to flanking sequences derived from HAstV ORF 2 and a 3′ untranslated region (3′ UTR) adjacent to ORF 2. FIG. 1C shows the expression of nLUC from the replicons described in FIG. 1B in 293T cells transfected with 100 ng replicon and harvested 24 hours later along with alphavirus replicons encoding nLUC or Zika virus (ZIKV) antigens as positive and negative controls, respectively.

FIGS. 2A-G show a comparison between nanostructured lipid-formulated replicons in the ability to induce immune responses in C57Bl/6 mice injected intramuscularly. FIG. 2A shows a schematic of an alphavirus-derived replicon featuring a subgenome encoding a gene of interest (GOI). FIG. 2B shows the results of a plaque reduction neutralization test for the alphavirus replicon with a ZIKV gene, at four dosages of NLC-formulated replicon as well as naked replicon and mock injection controls. FIG. 2C shows percentages of two different antigen-specific CD8 T-cells induced by the alphavirus replicon. FIG. 2D shows levels of two different antigen-specific CD4 T-cells induced by the alphavirus replicon. FIG. 2E shows a schematic of one of the astrovirus replicons from FIG. 1B (5′-HAstV-3′) with its subgenome encoding a GOI.

FIG. 2F shows combined percentages of antigen-specific CD4 T-cells induced by alphavirus and astrovirus replicons encoding two GOIs: ZIKV NS3 antigen and Mycobacterium tuberculosis antigen ID-93. Also shown are results from ID-93 protein alone and with GLA-SE adjuvant. FIG. 2G shows anti-hemagglutinin immunoglobulin G ELISA titers after a single dose of an unadjuvanted linear epitope (PR8 hemaagglutinin (HA) subunit) to naive mice and mice previously primed with an astrovirus replicon encoding a partial sequence of the hemagglutinin gene.

FIGS. 3A-D show effects of ORF 2 sequence length on predicted replicon secondary RNA structure and on gene expression. FIG. 3A shows the predicted secondary structure of a 400-nucleotide (nt) region encompassing the 3′ end of ORF 1b and the 5′ end ORF 2 of wild-type (WT) HAstV. The triple-hairpin structure is depicted in light and medium weight lines with the ORF 2 start codon boxed. FIG. 3B shows the predicted secondary structure of a 167-nt region in the 5′-3′ HAstV replicon containing the first 9 nt of ORF 2 depicting the apparent loss of the triple-hairpin structure. FIG. 3C shows the predicted secondary structure of a 188-nt region that includes the first 30 nt of ORF 2 (depicted in light weight lines on the lower right of the structure) with the apparent stabilization of the triple-hairpin structure. FIG. 3D shows results of a luciferase assay in 293T cells for three of the replicons from FIG. 1B with the first 9 nt of ORF 2 and three replicons including the first 30 nt of ORF 2: with unmodified flanking sequences (5′-30 nt-3′); modified 5′ flanking sequence (Δ5′-30 nt-3′); and unmodified flanking sequences with addition of a Thosea asigna virus 2A (T2A) ribosomal skipping sequence (5′-30 nt-T2A-3′).

FIGS. 4A-C show the results of coupled transcription and translation assays to detect subgenome transcription and translation in alphavirus and astrovirus replicons. FIG. 4A shows subgenome transcription in CaCo-2 cells infected with wild-type HAstV. FIG. 4B shows quantitative reverse transcription polymerase chain reaction (qRT-PCR) and luciferase assay results following transfection of 293T cells with the 5′-30 nt-T2A-3′ replicon encoding nLUC as well as uncapped controls. FIG. 4C shows qRT-PCR and luciferase assay results following transfection of 293T cells with an alphavirus replicon encoding nLUC as well as uncapped controls.

FIG. 5 shows a comparison of gene expression in two types of cells between 5′-30 nt-T2A-3′ replicons in which the hypervariable region (HVR) of ORF 1a is of the native genotype as (HVR-VII) or a divergent genotype (HVR-IV).

FIGS. 6A-B show the results of a bicistronic reporter assay used to investigate ORF 2 translational control mechanisms, particular translational termination-reinitiation. FIG. 6A shows a summary of selected mutations of ORF 1b. FIG. 6B shows the results of a dual luciferase assay of BHK cell lysates 24 hours after transfection with 100 ng of each plasmid described in FIG. 6A.

FIGS. 7A-B show the dependence of downstream ORF expression on the upstream ORF sequence.

FIG. 7A shows a depiction of plasmid constructs with deletions in ORF 1b. FIG. 7B shows the results of a dual luciferase assay in BHK cells 24 hours after transfection with 100 ng of each plasmid.

### DETAILED DESCRIPTION

As used herein, “replicon nucleic acid” or “replicon” refers to a ribonucleic acid (RNA) molecule, or a region of RNA, that replicates from a single origin of replication. The term “recombinant replicon nucleic acid” refers to a replicon nucleic acid that has been altered through human intervention. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

The term “nucleic acid sequence” refers to the sequence of a nucleic acid molecule. The nomenclature for nucleotide bases as set forth in 37 C.F.R. § 1.822 is used herein. Nucleic acid molecules can be any length, including but not limited to, between 3 Kb and 50 Kb, for example between 3 Kb and 40 Kb, between 3 Kb and 40 Kb, between 3 Kb and 30 Kb, between 3 Kb and 20 Kb, between 5 Kb and 40 Kb, between 5 Kb and 40 Kb, between 5 Kb and 30 Kb, between 5 Kb and 20 Kb, or between 10 Kb and 50 Kb, for example between 15 Kb to 30 Kb, between 20 Kb and 50 Kb, between 20 Kb and 40 Kb, 5 Kb and 25 Kb, or 30 Kb and 50 Kb. The nucleic acid molecules can also be, for example, more than 50 kb.

The term “open reading frame” (ORF) means a nucleic acid sequence consisting of a continuous stretch of codons that begins with a start codon (typically AUG) and ends at a stop codon (typically UAA, UAG or UGA). More than one open reading frame may be present in a single nucleic acid molecule, and one open reading frame may overlap another open reading frame on the same molecule. For example, the nucleic acid sequence of one open reading frame may include the start codon for another open reading frame.

The term “an astrovirus 5′ untranslated region (5′ UTR)” means a fragment of the astrovirus genome comprising the nucleic acid sequence located upstream of the initiating AUG of the open reading frame ORF 1a.

“An astrovirus 3′ untranslated region (3′ UTR)” means a fragment of the astrovirus genome comprising the nucleic acid sequence located downstream of the termination codon of the open reading frame ORF 2.

A “subgenomic promoter” is a promoter that directs transcription of a subgenomic messenger RNA as part of the replication process. Such a promoter can have a wild type sequence or a sequence that has been modified from wild type sequence but retains promoter activity.

The term “a conserved sequence element (CSE)” describes an RNA element that has a similar position, sequence, and secondary structure in the genomes of all of the known human astroviruses.

An “isolated cell” as used herein is a cell or population of cells that have been removed from the environment in which the cell occurs naturally and/or altered or modified from the state in which the cell occurs in its natural environment. An isolated cell can be a cell, for example, in a cell culture. An isolated cell can also be a cell that can be in an animal and/or introduced into an animal and wherein the cell has been altered or modified, e.g., by the introduction into the cell of an alphavirus particle.

A “subject” includes, but is not limited to, warm-blooded animals, e.g., humans, non-human primates, horses, cows, cats, dogs, pigs, rats, and mice.

Some embodiments provide a composition (e.g., a pharmaceutical composition) comprising a replicon encapsulated in a supramolecular structure to form a nanoparticle, where a plurality of such nanoparticles are dispersed in a pharmaceutically acceptable carrier. In particular embodiments, the nanoparticle comprises a replicon encapsulated in a lipid-based nanoparticle. In a specific aspect, the nanoparticle comprises a replicon encapsulated in a nanostructured lipid carrier (NLC). An example of one suitable NLC is described in Erasmus et al., Molecular Therapeutics 26(10):2507-2522 (2018).

By “pharmaceutically acceptable” is meant a material that is not biologically or otherwise undesirable, i.e., the material may be administered to a subject along with the selected nanoparticles, without causing substantial deleterious biological effects or interacting in a deleterious manner with any of the other components of the composition in which it is contained. The pharmaceutically acceptable carrier is suitable for administration or delivery to humans and other subjects. The carrier would naturally be selected to minimize any degradation of the active ingredient and to minimize any adverse side effects in the subject, as would be well known to one of skill in the art (see, e.g., Remington's Pharmaceutical Science; latest edition). Pharmaceutical formulations, such as vaccines or other immunogenic compositions can comprise an immunogenic amount of the astrovirus replicons disclosed, in combination with a pharmaceutically acceptable carrier. Exemplary pharmaceutically acceptable carriers include, but are not limited to, sterile pyrogen-free water and sterile pyrogen-free physiological saline solution.

Administration of the various compositions (e.g., nucleic acids, nanoparticles, pharmaceutical compositions) can be accomplished by any of several different routes. The compositions can be administered intramuscularly, subcutaneously, intraperitoneally, intradermally, intranasally, intracranially, sublingually, intravaginally, intrarectally, orally, or topically. The compositions can also be administered via a skin scarification method, or transdermally via a patch or liquid. The compositions can also be delivered subdermally in the form of a biodegradable material that releases the compositions over a period of time. The compositions can also be delivered intramuscularly via injection.

The nucleic acids, nanoparticles, and pharmaceutical compositions can be employed in methods of delivering a secreted protein of interest to a cell, which can be a cell in a subject. Thus, some embodiments provide a method of introducing into a cell an effective amount of a nucleic acid, nanoparticle and/or composition of the embodiments. Also provided is a method of delivering to the subject an effective amount of a nucleic acid, nanoparticle and/or composition of the embodiments. Such methods can be employed to impart a therapeutic effect on a cell and/or a subject, according to well-known protocols for gene therapy.

Astrovirus replicons provide an attractive alternative by combining their smaller genome size with those features provided by alphavirus replicons: a subgenomic RNA replication strategy and delayed yet robust induction of IFN. Astrovirus replicon machinery is encoded by a ˜4 kb RNA while those of alphavirus or flavivirus origin are encoded by an ˜8 kb RNA. This reduces the effective dose in terms of copy-number by roughly 2-fold.

As used herein, “effective amount” refers to an amount of a composition or formulation that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science and Practice of Pharmacy (20th ed. 2000)).

The replicon RNA compositions described herein are administered in a manner compatible with the dosage formulation, and in such amount as will be prophylactically and/or therapeutically effective. The quantity to be administered, which can generally be in the range of 10⁴ to 10¹⁰ units in a dose (e.g., 10⁴, 10⁵, 10⁶, 10⁷, 10⁸, 10⁹, or 10¹⁰), depends on the subject to be treated, the route by which the particles are administered or delivered, the immunogenicity of the expression product, the types of effector immune responses desired, and the degree of protection desired. Effective amounts of the active ingredient required to be administered or delivered may depend on the judgment of the physician, veterinarian or other health practitioner and may be specific for a given subject, but such a determination is within the skill of such a practitioner.

The compositions and formulations disclosed may be given in a single dose or a multiple dose schedule. A multiple dose schedule is one in which a primary course of administration may include 1 to 10 or more separate doses, followed by other doses administered at subsequent time intervals as required to maintain and or reinforce the desired effect (e.g., a therapeutic response).

“Therapeutic amount” refers to an amount sufficient to impart a modulating effect (e.g., a therapeutic response), which, for example, can be a beneficial effect to a subject afflicted with a disorder, disease or illness, including improvement in the condition of the subject (e.g., in one or more symptoms), delay or reduction in the progression of the condition, delay of the onset of the disorder, disease or illness, and/or change in any of the clinical parameters of a disorder, disease or illness, etc., as would be well known in the art. Another example of therapeutic response contemplated in this disclosure is an increased resistance to a pathogenic disease through stimulation of the subject's immune system.

As used herein, “a,” “an” and “the” can mean one or more than one, depending on the context in which it is used. For example, “a” cell can mean one cell or multiple cells. Also, as used herein, “and/or” refers to and encompasses any and all possible combinations of one or more of the associated listed items, as well as the lack of combinations when interpreted in the alternative (“or”).

It is understood that the foregoing detailed description is given merely by way of illustration and that modifications and variations may be made therein without departing from the spirit and scope of the invention.

### EXAMPLES

The following examples are provided to illustrate certain disclosed embodiments and are not to be construed as limiting the scope of this disclosure in any way.

### Example 1—Materials and Methods

### Preparation of Replicon RNA

Various astrovirus replicon sequences were synthesized and cloned into plasmids downstream of a T7 promoter and upstream of a hepatitis delta virus ribozyme sequence, T7 terminator, and NotI restriction site. To prepare RNA for downstream studies, purified plasmids were linearized by restriction digest with NotI enzyme followed by purification by phenol-chloroform and ethanol precipitation. Linearized template was then used for transcription of RNA using T7 polymerase and purified by LiCl precipitation and ethanol wash. RNA transcripts were then capped using Vaccinia virus capping enzyme and purified by LiCl precipitation and ethanol wash.

### Example 2—Protein Expression from Replicons Having Selected Modifications

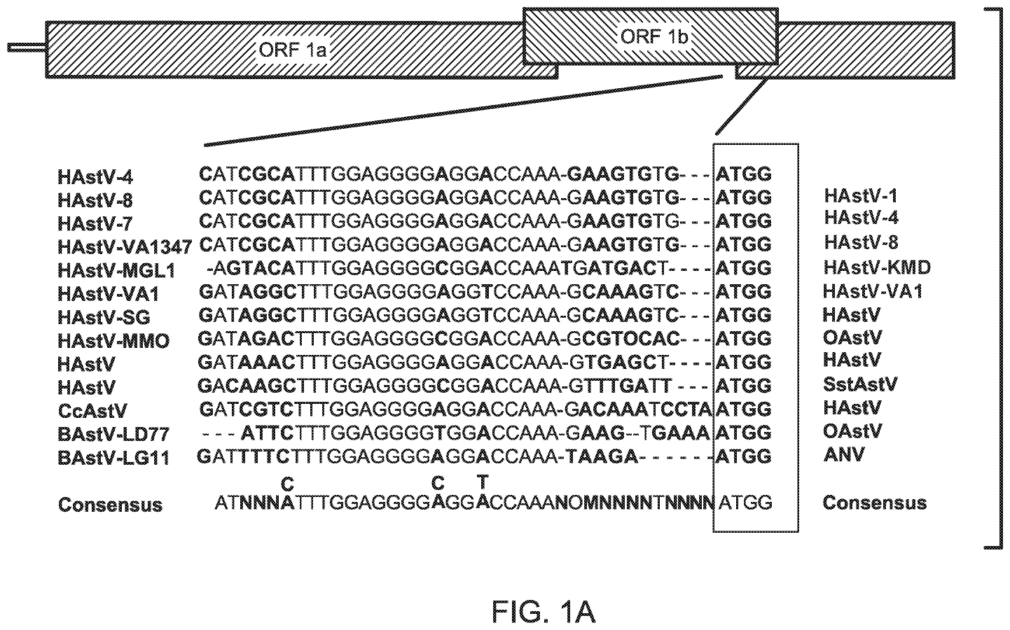

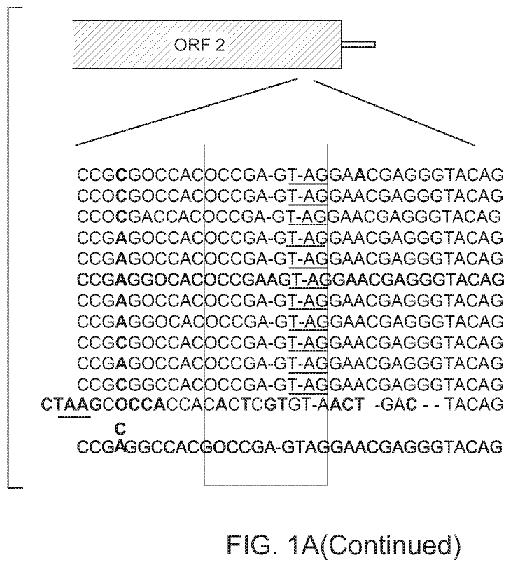

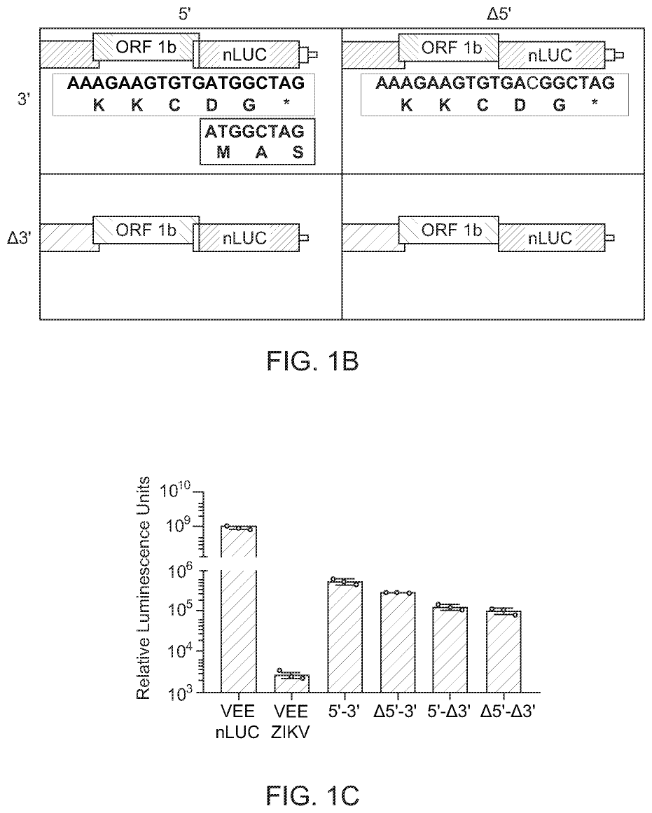

Wild-type (WT) Human astrovirus (HAstV) replication machinery consists of a 5′ and 3′ untranslated region (UTR) as well as nonstructural proteins, nsP1a and nsP1b, respectively encoded by two overlapping open reading frames, ORF1a and ORF1b, processed by a ribosomal frame-shift mechanism. The 3′ end of ORF 1b contains a proposed subgenomic promoter that is hypothesized to initiate the transcription of a subgenomic RNA, mediated by the proteins translated from ORFs 1a and 1b. The structural proteins, encoded by ORF 2, are thought to be translated from this subgenomic RNA whose initiation codon overlaps with the 3′ end of ORF 1b. Additionally, a highly conserved stem loop sequence is present beginning at the 3′ end of ORF 2 and ending in the 3′ UTR. See FIG. 1A.

Four replicons containing a combination of these conserved sequence elements to test whether they were important for ORF 2 expression were developed. The 5′-3′ replicon contained intact 5′ and 3′ conserved sequence elements. Other replicons contained a synonymous mutation in ORF1b that silenced the initiating methionine of ORF 2 (Δ5′), a deletion of the conserved 3′ ORF 2 sequence (Δ3′), or both (Δ5′-Δ3′). ORF 2 included a sequence encoding NanoLuc® luciferase (nLUC). See FIG. 1B.

To test the effect of the sequence elements on ORF 2 expression, 293T cells were transfected with 100 ng of each replicon along with alphavirus replicons encoding nLUC or Zika virus (ZIKV) antigens as positive and negative controls, respectively, and nLUC expression was measured 24 hours later. The results of this test are shown in FIG. 1C. While intact 5′ and 3′ sequences enhanced nLUC expression 5-fold over the Δ5′-Δ3′ counterpart and about 25-fold over background, expression levels were ˜17,000-fold below that of the alphavirus positive control, suggesting that additional nucleic acid sequence are likely important in enhancing ORF 2 expression.

### Example 3—CD4+ T-cell Induction by Recombinant Astrovirus Replicons

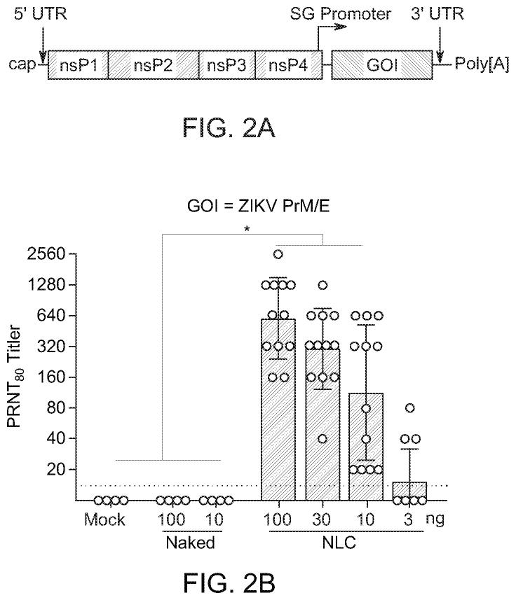

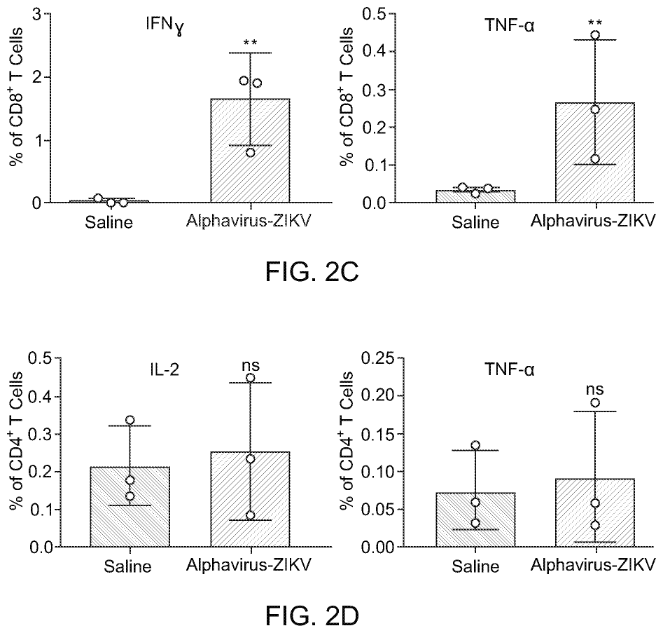

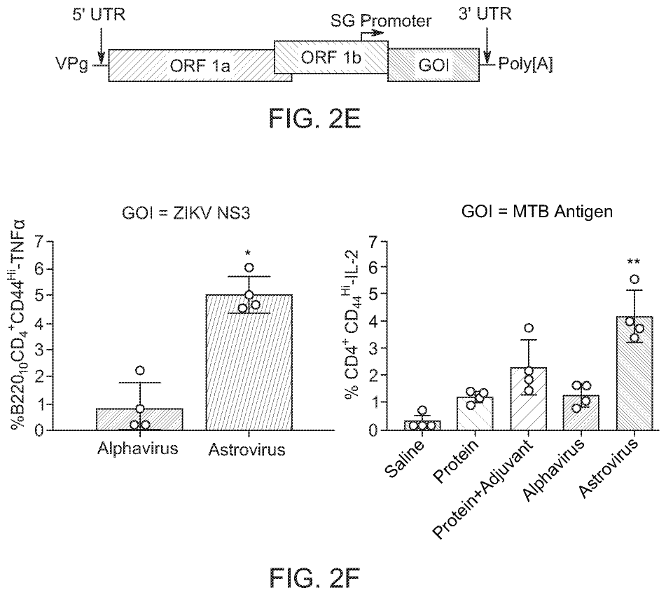

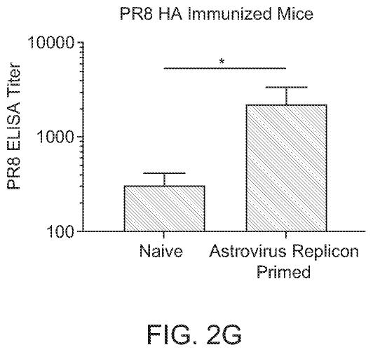

Next the ability of the 5′-3′ HAstV replicon to induce T-cell responses to a Mycobacterium tuberculosis antigen, ID-93, or to Zika virus NS3 antigen was assessed. To prepare these constructs, sequences encoding ID-93 or Zika virus NS3 proteins were synthesized and cloned into the 5′-3′ HAstV replicon (FIG. 2E) between AvrII and PvuI restriction sites and also into an alphavirus replicon derived from TC-83 strain of Venezuelan equine encephalitis virus (FIG. 2A) between PfIMI and SacII restriction sites using Gibson assembly. Capped RNAs were then prepared as described in Example 1 above and transfected into BHK cells to confirm antigen expression by western blot (data not shown). Following confirmation of antigen expression, replicons were formulated in nanostructured lipid carriers and 1 μg of each replicon was administered via a single intramuscular injection into C57BL/6 mice. For the ID-93 replicons, additional controls of protein subunit alone as well as adjuvanted (GLA-SE) protein subunit were included to compare T-cell responses between replicons and a more traditional vaccine preparation, the latter of which has been previously shown to induce potent antigen-specific CD4⁺ T-cell responses in mice. Fourteen days after a single injection, spleens were harvested and stimulated with MHC-II restricted peptides derived from either Zika virus NS3 or Mycobacterium ID-93 antigens and stained for analysis by flow cytometry (FIG. 2F).

The results, shown in FIG. 2, demonstrated that while non-viral delivery of alphavirus-derived replicons encoding bacterial or viral antigens drive potent CD8+ and antibody responses to the encoded antigens following a single intramuscular injection (FIG. 2 B-D), astrovirus-derived replicons encoding the same antigens drive significantly higher antigen-specific CD4+ T-cell responses (FIG. 2F).

To test whether these CD4+ T-cell responses to linear epitopes could enhance antibody responses to whole protein subunits, C57Bl/6 mice were primed with a single dose of an astrovirus replicon encoding a 15 amino acid (aa) sequence of the hemagglutinin (HA) gene conserved amongst seasonal influenza virus subtypes and previously shown to be reactive in C57Bl/6 mice. Twenty-one days later a single dose of unadjuvanted recombinant PR8 HA subunit protein was administered to astrovirus RNA-primed as well as naïve mice and compared anti-HA IgG ELISA titers. Mice primed with the astrovirus RNA encoding the conserved 15 aa sequence mounted significantly higher (5.5-fold) anti-HA ELISA titers (mean=1:2200) compared to mice receiving protein alone (mean=1:400) (FIG. 2G).

### Example 4—Effect of ORF 2 Sequence Composition on Expression and Translation

### A. ORF 2 Sequence Length

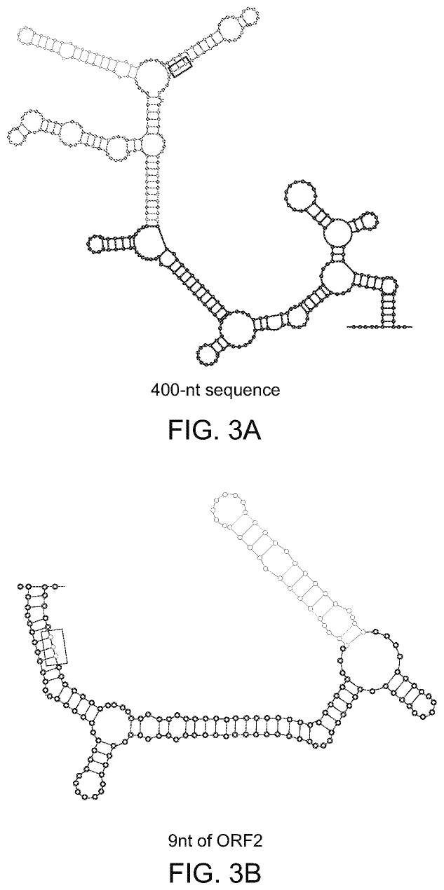

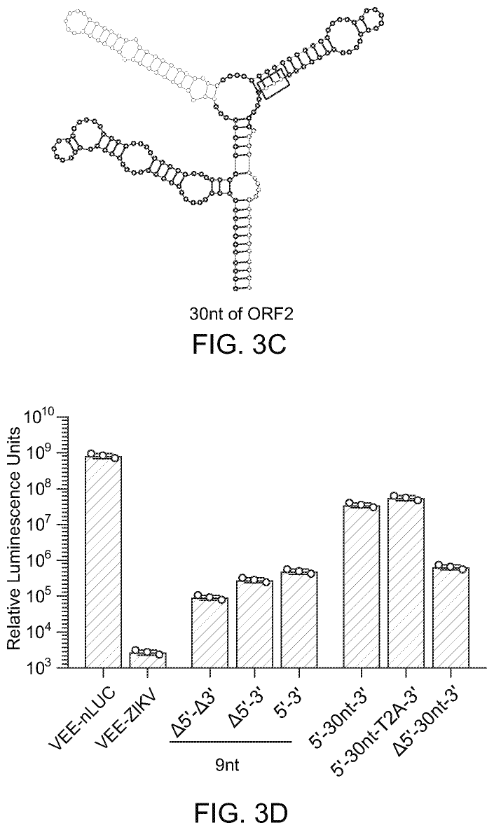

Predicted secondary RNA structures were assessed for a 400-bp region in WT HAstV that included the proposed subgenomic RNA promoter and compared that with the predicted structure for the 5′-3′ nLUC replicon of Examples 2 and 3, which contains the first 9 nucleotides (nt) of ORF 2. In the WT sequence (shown in FIG. 3A), a triple-hairpin structure is clearly observed (represented as a structure rendered with light and medium weight lines), with the start codon for ORF 2 boxed. In the predicted secondary structure for the 5′-3′ replicon with the first 9 nt of ORF 2 (shown in FIG. 3B), two hairpin structures are not present with only the hairpin (depicted in light weight lines), conserved between WT and replicon sequences. By including an additional 21 nt from ORF 2, depicted in light weight lines on the lower right of the diagram, the triple-hairpin structure appears to become stabilized in the prediction (FIG. 3C).

To assess the role of these 30 nt in HAstV replication, additional HAstV replicons were constructed containing the 30-nt sequence with or without the synonymous mutation in ORF 1b (5′-30 nt-3′ or Δ5′-30 nt-3′ replicons, respectively). For the 5′-30 nt-3′ replicon which encodes the N-terminal 10 amino acids of ORF 2, a Thosea asigna virus 2A (T2A) ribosomal skipping sequence was inserted before the nLUC gene to make the 5′-30 nt-T2A-3′ replicon.

Following in vitro transcription and capping of RNA, 293T cells were transfected with each construct at the same dose, including two Venezuelan equine encephalitis virus replicons, one including a gene for nLUC (VEE-nLUC) and the other including a gene for ZIKV antigen (VEE-ZIKV) as positive and negative controls, respectively. At 24 hours after transfection, a luciferase assay was performed to quantify heterologous gene expression. The results, shown in FIG. 3D, support the conclusion that the 30 nt enhance heterologous gene expression and that the ORF 2 start codon is involved in efficient translation initiation, with luciferase activity detected at over 16,000-fold above background levels, approaching within 30-fold of the alphavirus replicating viral RNA.

### B. Role of Identified ORF 2 Sequence on Translation

Having demonstrated the importance of the first 30 nt in ORF 2 expression, next the role of this sequence element in subgenome transcription was determined.

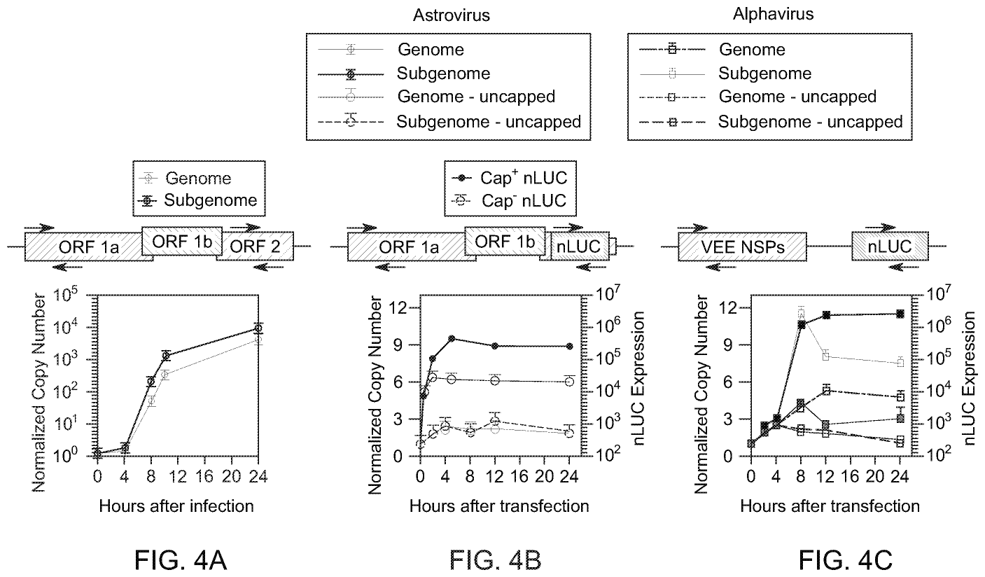

A quantitative reverse-transcription (qRT) PCR assay was designed to quantify genome and subgenome copies that accumulate during astrovirus replication and validated the assay in the context of WT astrovirus replication. CaCo-2 cells were infected with WT astrovirus at a multiplicity of infection of 0.1 and harvested cell lysates at 0, 4, 8, 12, and 24 hours post-infection. RNA was then extracted and run in the qRT-PCR assay along with T7-transcribed RNA from the infectious clone of the same virus to be used as a standard curve. Subgenome transcription could be detected at a 5-fold excess compared to genome transcription beginning at 8 hours after infection (FIG. 4A), re-capitulating previously published northern blot data and confirming that this assay can indeed detect subgenome transcription.

Next, this assay was applied in the context of replicons which do not encode the structural genes and cannot spread between cells and would also allow for simple quantification of ORF 2 expression coupled with transcription. As a positive control, a similar qRT-PCR assay to detect genome and subgenome of an alphavirus replicon was designed. While the alphavirus replicon demonstrated excess subgenome transcription beginning at 8 hours after transfection, coinciding with nLUC expression (FIG. 4B), the astrovirus replicon (5′-30 nt-T2A-3′) demonstrated no evidence of subgenome transcription in excess of genome, and interestingly, nLUC expression could be detected as early as 30 min after transfection (FIG. 4B).

These results suggest that: 1) the tested sequence elements are insufficient for mediating subgenomic RNA transcription, and 2) HAstV utilizes an alternative mechanism of ORF 2 expression that allows for early translation of ORF 2 independently of subgenomic RNA transcription. Similar observations have been made for caliciviruses which also utilize a subgenomic message to translate their structural genes. Early expression of structural genes independent of subgenome transcription in bovine norovirus have been described. This may also suggest an important role for structural gene expression in RNA replication.

### Example 5—Effects of ORF 1a Chimerism on ORF 2 Gene Expression

A region of ORF 1a, termed the hypervariable region (HVR), that is associated with differences in genome and subgenome transcription was examined. An HVR derived from genotype IV HAstVs is associated with higher titers of virus in clinical samples as well as differences in subgenome to genome ratios.

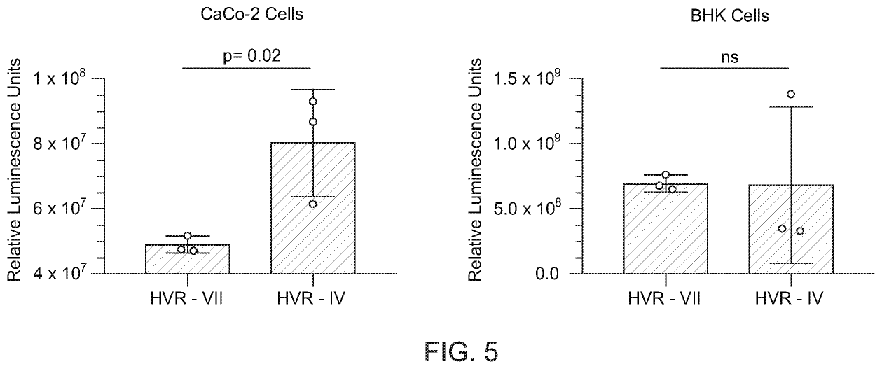

Using the 5′-30 nt-T2A-3′ replicon described above as the backbone (genotype VII HVR), the HVR was replaced with that of a genotype IV HAstV and transfected CaCo2 as well as BHK cells. As shown in FIG. 5, while no difference in ORF 2 expression was detected in BHK cells, a small yet significant difference was detected on CaCo-2 cells, supporting the previously published data observed for wt virus on CaCo-2 cells. The disparity in results between cell lines suggests a role for the HVR in host-range. Interestingly, the HVR chimera demonstrated lower toxicity in E. coli resulting in higher plasmid yields which may prove useful in downstream applications of astrovirus replicons.

### Example 6—Mechanisms of Subgenome Transcription-Independent ORF 2 Translation

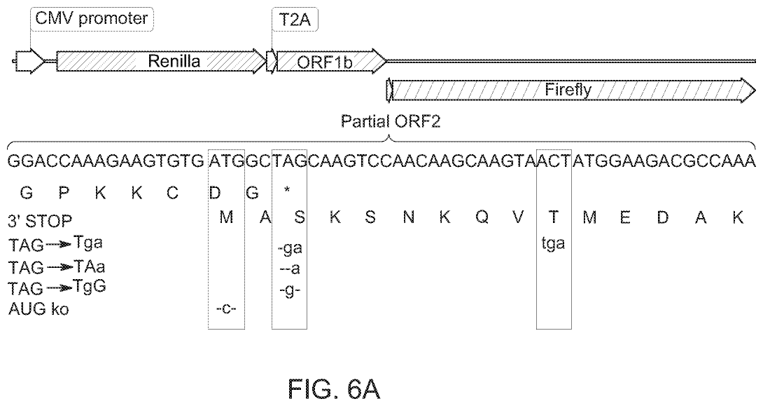

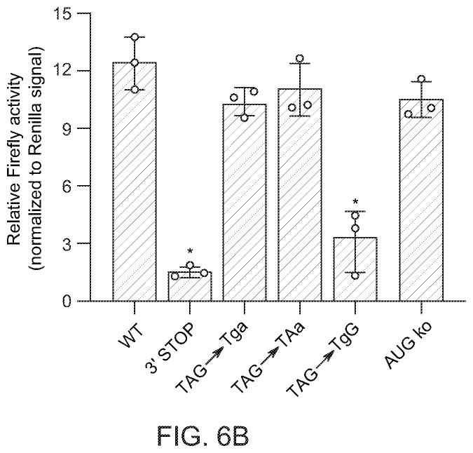

Given the evolutionary relationship between caliciviruses and astroviruses, it was next tested whether astroviruses utilize translation termination reinitiation (TTR) between ORF 1b and ORF 2 in a similar manner to caliciviruses, allowing for subgenome transcription-independent translation of ORF 2 early in the replication cycle. To test this hypothesis, a bicistronic reporter was generated encoding Renilla and Firefly luciferases in the first and second ORFs respectively, separated by an 808 bp region of ORF 1b and ORF 2 of HAstV-1 (FIG. 6A). Then a series of mutations were made to test the importance of the ORF 1b stop codon as well as the ORF 2 start codons because TTR in caliciviruses has been shown to be dependent on the location of the upstream ORF stop codon but not on the downstream ORF start codon. As a negative control, a stop codon was inserted at the end of ORF 1b immediately prior to the ORF 2 initiating codon (3′ STOP). Following transfection of BHK cells with 100 ng of each plasmid, a dual luciferase assay was performed to first measure Firefly activity, followed by quenching and detection of Renilla activity (FIG. 6B). Downstream ORF (Firefly) expression, relative to upstream ORF (Renilla) expression, was then normalized to the negative control (3′ STOP).

WT ORF1b/ORF 2 sequence resulted in a 12-fold increase in downstream ORF expression relative to the 3′ STOP negative control. While the type of the ORF 1b stop codon does not appear to be important for downstream ORF expression, changing the location by replacing the stop codon with TGG coding for tryptophan, resulting in an extension of the ORF 1b reading frame an additional 34 codons before terminating, appears to abolish downstream ORF expression. Finally, replacing the start codon of ORF 2 with ACG appears to not affect downstream ORF 2 expression. These findings are consistent with TTR in caliciviruses.

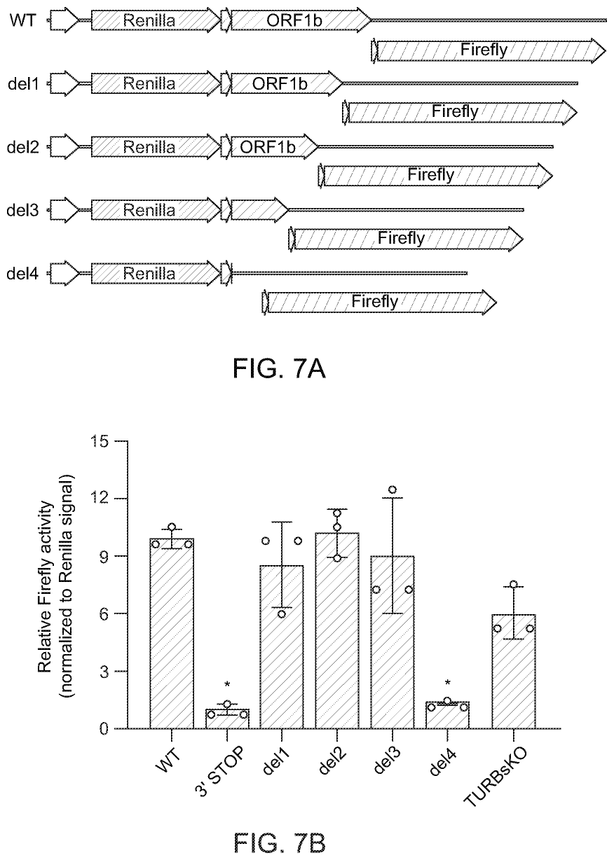

The mechanism of TTR in caliciviruses has been shown to depend on complementary sequence in host 18s ribosomal RNA binding an upstream sequence, termed a translational upstream ribosome binding site (TURBS), in the calicivirus genome, allowing for disengaged ribosomes to reinitiate translation of the downstream ORF. To identify the potential location of such a sequence in astrovirus ORF1b, next a series of deletion mutants in the bicistronic reporter system (FIG. 7A) were generated. Additionally, mutations were also generated within a TURBS-like sequence located within the del3 mutant to see whether that sequence was important for downstream ORF expression. The results suggest that while the TURBS-like sequence did not seem to significantly affect downstream ORF expression, the 200 bp sequence located within the del3 mutant is required (FIG. 7B). Interestingly, this 200 bp sequence is predicted to form the triple hairpin structure depicted in FIG. 3C.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term refers to a numeric value, including, for example, whole numbers, fractions, and percentages, whether or not explicitly indicated. The term generally refers to a range of numerical values (e.g., +/−5-10% of the recited range) that one of ordinary skill in the art would consider equivalent to the recited value (e.g., having the same function or result). When terms such as at least and precede a list of numerical values or ranges, the terms modify all of the values or ranges provided in the list. In some instances, the term may include numerical values that are rounded to the nearest significant FIG..

### SEQUENCE TABLE

The following sequence table provides a listing of sequences disclosed herein. It is understood that if a DNA sequence (comprising Ts) is referenced with respect to an RNA, then Ts should be replaced with Us (which may be modified or unmodified depending on the context), and vice versa.

| Description | Sequence | SEQ ID NO: |
|---|---|---|
| Transcript of 5′- | CCAAGAGGGGGGTGGTGATTGGCCTTTGGCTTATCAGTGTATA | 1 |
| Δ3′ HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGATGGCTAGCCCTAG | |
| | GATGGTCTTCACACTCGAAGATTTCGTTGGGGACTGGCGACAG | |
| | ACAGCCGGCTACAACCTGGACCAAGTCCTTGAACAGGGAGGTG | |
| | TGTCCAGTTTGTTTCAGAATCTCGGGGTGTCCGTAACTCCGAT | |
| | CCAAAGGATTGTCCTGAGCGGTGAAAATGGGCTGAAGATCGAC | |
| | ATCCATGTCATCATCCCGTATGAAGGTCTGAGCGGCGACCAAA | |
| | TGGGCCAGATCGAAAAAATTTTTAAGGTGGTGTACCCTGTGGA | |
| | TGATCATCACTTTAAGGTGATCCTGCACTATGGCACACTGGTA | |
| | ATCGACGGGGTTACGCCGAACATGATCGACTATTTCGGACGGC | |
| | CGTATGAAGGCATCGCCGTGTTCGACGGCAAAAAGATCACTGT | |
| | AACAGGGACCCTGTGGAACGGCAACAAAATTATCGACGAGCGC | |
| | CTGATCAACCCCGACGGCTCCCTGCTGTTCCGAGTAACCATCA | |
| | ACGGAGTGACCGGCTGGCGGCTGTGCGAACGCATTCTGGCGTA | |
| | ATAGACGCGTGAACGAGGGTACAGCTTCCTTCTTTTCTGTCTC | |
| | TGTTTAGATTATTTTAATCACCATTTAAAATTGATTTAATCAG | |
| | AAGCAAAAAAAAAAAAAAAAAAAAAAAAAAAAAAAAAAAAAAA | |
| | AAAAAAGGGTCGGCATGGCATCTCCACCTCCTCGCGGTCCGAC | |
| | CTGGGCATCCGAAGGAGGACGCACGTCCACTCGGATGGCTAAG | |
| | GGAGCCTGCATTCGCAGAAGCCACCCGCGCTGCTAACAAAGCC | |
| | CGAAAGGAAGCTGAGTTGGCTGCTGCCACCGCTGAGCAATAAC | |
| | TAGCATAACCCCTTGGGGCCTCTAAACGGGTCTTGAGGGGTTT | |
| | TTTGCTGAAAGGAGGAACTATATGCGCTCATA | |
| Transcript of Δ5′- | CCAAGAGGGGGGTGGTGATTGGCCTTTGGCTTATCAGTGTATA | 2 |
| Δ3′ HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGACGGCTAGCCCTAG | |
| | GATGGTCTTCACACTCGAAGATTTCGTTGGGGACTGGCGACAG | |
| | ACAGCCGGCTACAACCTGGACCAAGTCCTTGAACAGGGAGGTG | |
| | TGTCCAGTTTGTTTCAGAATCTCGGGGTGTCCGTAACTCCGAT | |
| | CCAAAGGATTGTCCTGAGCGGTGAAAATGGGCTGAAGATCGAC | |
| | ATCCATGTCATCATCCCGTATGAAGGTCTGAGCGGCGACCAAA | |
| | TGGGCCAGATCGAAAAAATTTTTAAGGTGGTGTACCCTGTGGA | |
| | TGATCATCACTTTAAGGTGATCCTGCACTATGGCACACTGGTA | |
| | ATCGACGGGGTTACGCCGAACATGATCGACTATTTCGGACGGC | |
| | CGTATGAAGGCATCGCCGTGTTCGACGGCAAAAAGATCACTGT | |
| | AACAGGGACCCTGTGGAACGGCAACAAAATTATCGACGAGCGC | |
| | CTGATCAACCCCGACGGCTCCCTGCTGTTCCGAGTAACCATCA | |
| | ACGGAGTGACCGGCTGGCGGCTGTGCGAACGCATTCTGGCGTA | |
| | ATAGACGCGTGAACGAGGGTACAGCTTCCTTCTTTTCTGTCTC | |
| | TGTTTAGATTATTTTAATCACCATTTAAAATTGATTTAATCAG | |
| | AAGCAAAAAAAAAAAAAAAAAAAAAAAAAAAAAAAAAAAAAAA | |
| | AAAAAAGGGTCGGCATGGCATCTCCACCTCCTCGCGGTCCGAC | |
| | CTGGGCATCCGAAGGAGGACGCACGTCCACTCGGATGGCTAAG | |
| | GGAGCCTGCATTCGCAGAAGCCACCCGCGCTGCTAACAAAGCC | |
| | CGAAAGGAAGCTGAGTTGGCTGCTGCCACCGCTGAGCAATAAC | |
| | TAGCATAACCCCTTGGGGCCTCTAAACGGGTCTTGAGGGGTTT | |
| | TTTGCTGAAAGGAGGAACTATATGCGCTCATA | |
| Transcript of 5′-3′ | CCAAGAGGGGGGTGGTGATTGGCCTTTGGCTTATCAGTGTATA | 3 |
| HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGATGGCTAGCCCTAG | |
| | GATGGTCTTCACACTCGAAGATTTCGTTGGGGACTGGCGACAG | |
| | ACAGCCGGCTACAACCTGGACCAAGTCCTTGAACAGGGAGGTG | |
| | TGTCCAGTTTGTTTCAGAATCTCGGGGTGTCCGTAACTCCGAT | |
| | CCAAAGGATTGTCCTGAGCGGTGAAAATGGGCTGAAGATCGAC | |
| | ATCCATGTCATCATCCCGTATGAAGGTCTGAGCGGCGACCAAA | |
| | TGGGCCAGATCGAAAAAATTTTTAAGGTGGTGTACCCTGTGGA | |
| | TGATCATCACTTTAAGGTGATCCTGCACTATGGCACACTGGTA | |
| | ATCGACGGGGTTACGCCGAACATGATCGACTATTTCGGACGGC | |
| | CGTATGAAGGCATCGCCGTGTTCGACGGCAAAAAGATCACTGT | |
| | AACAGGGACCCTGTGGAACGGCAACAAAATTATCGACGAGCGC | |
| | CTGATCAACCCCGACGGCTCCCTGCTGTTCCGAGTAACCATCA | |
| | ACGGAGTGACCGGCTGGCGGCTGTGCGAACGCATTCTGGCGTA | |
| | ATAGACGCGTgcaaagcagcaggggaaatcaatccctgcacat | |
| | ctggaagccgcggccacgccgagtagGAACGAGGGTACAGCTT | |
| | CCTTCTTTTCTGTCTCTGTTTAGATTATTTTAATCACCATTTA | |
| | AAATTGATTTAATCAGAAGCAAAAAAAAAAAAAAAAAAAAAAA | |
| | AAAAAAAAAAAAAAAAAAAAAAGGGTCGGCATGGCATCTCCAC | |
| | CTCCTCGCGGTCCGACCTGGGCATCCGAAGGAGGACGCACGTC | |
| | CACTCGGATGGCTAAGGGAGCCTGCATTCGCAGAAGCCACCCG | |
| | CGCTGCTAACAAAGCCCGAAAGGAAGCTGAGTTGGCTGCTGCC | |
| | ACCGCTGAGCAATAACTAGCATAACCCCTTGGGGCCTCTAAAC | |
| | GGGTCTTGAGGGGTTTTTTGCTGAAAGGAGGAACTATATGCGC | |
| | TCATA | |
| Transcript of Δ5′- | CCAAGAGGGGGGTGGTGATTGGCCTTTGGCTTATCAGTGTATA | 4 |
| 3′ HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGACGGCTAGCCCTAG | |
| | GATGGTCTTCACACTCGAAGATTTCGTTGGGGACTGGCGACAG | |
| | ACAGCCGGCTACAACCTGGACCAAGTCCTTGAACAGGGAGGTG | |
| | TGTCCAGTTTGTTTCAGAATCTCGGGGTGTCCGTAACTCCGAT | |
| | CCAAAGGATTGTCCTGAGCGGTGAAAATGGGCTGAAGATCGAC | |
| | ATCCATGTCATCATCCCGTATGAAGGTCTGAGCGGCGACCAAA | |
| | TGGGCCAGATCGAAAAAATTTTTAAGGTGGTGTACCCTGTGGA | |
| | TGATCATCACTTTAAGGTGATCCTGCACTATGGCACACTGGTA | |
| | ATCGACGGGGTTACGCCGAACATGATCGACTATTTCGGACGGC | |
| | CGTATGAAGGCATCGCCGTGTTCGACGGCAAAAAGATCACTGT | |
| | AACAGGGACCCTGTGGAACGGCAACAAAATTATCGACGAGCGC | |
| | CTGATCAACCCCGACGGCTCCCTGCTGTTCCGAGTAACCATCA | |
| | ACGGAGTGACCGGCTGGCGGCTGTGCGAACGCATTCTGGCGTA | |
| | ATAGACGCGTgcaaagcagcaggggaaatcaatccctgcacat | |
| | ctggaagccgcggccacgccgagtagGAACGAGGGTACAGCTT | |
| | CCTTCTTTTCTGTCTCTGTTTAGATTATTTTAATCACCATTTA | |
| | AAATTGATTTAATCAGAAGCAAAAAAAAAAAAAAAAAAAAAAA | |
| | AAAAAAAAAAAAAAAAAAAAAAGGGTCGGCATGGCATCTCCAC | |
| | CTCCTCGCGGTCCGACCTGGGCATCCGAAGGAGGACGCACGTC | |
| | CACTCGGATGGCTAAGGGAGCCTGCATTCGCAGAAGCCACCCG | |
| | CGCTGCTAACAAAGCCCGAAAGGAAGCTGAGTTGGCTGCTGCC | |
| | ACCGCTGAGCAATAACTAGCATAACCCCTTGGGGCCTCTAAAC | |
| | GGGTCTTGAGGGGTTTTTTGCTGAAAGGAGGAACTATATGCGC | |
| | TCATA | |
| Transcript of 5′- | CCAAGAGGGGGGTGGTGATTGGCCTTTGGCTTATCAGTGTATA | 5 |
| 10AA-3′ HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGATGGCTAGCAAGTC | |
| | CAACAAGCAAGTAACTCCTAGGATGGTCTTCACACTCGAAGAT | |
| | TTCGTTGGGGACTGGCGACAGACAGCCGGCTACAACCTGGACC | |
| | AAGTCCTTGAACAGGGAGGTGTGTCCAGTTTGTTTCAGAATCT | |
| | CGGGGTGTCCGTAACTCCGATCCAAAGGATTGTCCTGAGCGGT | |
| | GAAAATGGGCTGAAGATCGACATCCATGTCATCATCCCGTATG | |
| | AAGGTCTGAGCGGCGACCAAATGGGCCAGATCGAAAAAATTTT | |
| | TAAGGTGGTGTACCCTGTGGATGATCATCACTTTAAGGTGATC | |
| | CTGCACTATGGCACACTGGTAATCGACGGGGTTACGCCGAACA | |
| | TGATCGACTATTTCGGACGGCCGTATGAAGGCATCGCCGTGTT | |
| | CGACGGCAAAAAGATCACTGTAACAGGGACCCTGTGGAACGGC | |
| | AACAAAATTATCGACGAGCGCCTGATCAACCCCGACGGCTCCC | |
| | TGCTGTTCCGAGTAACCATCAACGGAGTGACCGGCTGGCGGCT | |
| | GTGCGAACGCATTCTGGCGTAATAGACGCGTgcaaagcagcag | |
| | gggaaatcaatccctgcacatctggaagccgcggccacgccga | |
| | gtagGAACGAGGGTACAGCTTCCTTCTTTTCTGTCTCTGTTTA | |
| | GATTATTTTAATCACCATTTAAAATTGATTTAATCAGAAGCAA | |
| | AAAAAAAAAAAAAAAAAAAAAAAAAAAAAAAAAAAAAAAAAAA | |
| | GGGTCGGCATGGCATCTCCACCTCCTCGCGGTCCGACCTGGGC | |
| | ATCCGAAGGAGGACGCACGTCCACTCGGATGGCTAAGGGAGCC | |
| | TGCATTCGCAGAAGCCACCCGCGCTGCTAACAAAGCCCGAAAG | |
| | GAAGCTGAGTTGGCTGCTGCCACCGCTGAGCAATAACTAGCAT | |
| | AACCCCTTGGGGCCTCTAAACGGGTCTTGAGGGGTTTTTTGCT | |
| | GAAAGGAGGAACTATATGCGCTCATA | |
| Transcript of Δ5′- | CCAAGAGGGGGGTGGTGATTGGCCTTTGGCTTATCAGTGTATA | 6 |
| 10AA-3′ HAstV-nLUC | GATAACATTTCTCTGACCGTTTACCACACAATTAACAACAATA | |
| | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGACGGCTAGCAAGTC | |
| | CAACAAGCAAGTAACTCCTAGGATGGTCTTCACACTCGAAGAT | |
| | TTCGTTGGGGACTGGCGACAGACAGCCGGCTACAACCTGGACC | |
| | AAGTCCTTGAACAGGGAGGTGTGTCCAGTTTGTTTCAGAATCT | |
| | CGGGGTGTCCGTAACTCCGATCCAAAGGATTGTCCTGAGCGGT | |
| | GAAAATGGGCTGAAGATCGACATCCATGTCATCATCCCGTATG | |
| | AAGGTCTGAGCGGCGACCAAATGGGCCAGATCGAAAAAATTTT | |
| | TAAGGTGGTGTACCCTGTGGATGATCATCACTTTAAGGTGATC | |
| | CTGCACTATGGCACACTGGTAATCGACGGGGTTACGCCGAACA | |
| | TGATCGACTATTTCGGACGGCCGTATGAAGGCATCGCCGTGTT | |
| | CGACGGCAAAAAGATCACTGTAACAGGGACCCTGTGGAACGGC | |
| | AACAAAATTATCGACGAGCGCCTGATCAACCCCGACGGCTCCC | |
| | TGCTGTTCCGAGTAACCATCAACGGAGTGACCGGCTGGCGGCT | |
| | GTGCGAACGCATTCTGGCGTAATAGACGCGTgcaaagcagcag | |
| | gggaaatcaatccctgcacatctggaagccgcggccacgccga | |
| | gtagGAACGAGGGTACAGCTTCCTTCTTTTCTGTCTCTGTTTA | |
| | GATTATTTTAATCACCATTTAAAATTGATTTAATCAGAAGCAA | |
| | AAAAAAAAAAAAAAAAAAAAAAAAAAAAAAAAAAAAAAAAAAA | |
| | GGGTCGGCATGGCATCTCCACCTCCTCGCGGTCCGACCTGGGC | |
| | ATCCGAAGGAGGACGCACGTCCACTCGGATGGCTAAGGGAGCC | |
| | TGCATTCGCAGAAGCCACCCGCGCTGCTAACAAAGCCCGAAAG | |
| | GAAGCTGAGTTGGCTGCTGCCACCGCTGAGCAATAACTAGCAT | |
| | AACCCCTTGGGGCCTCTAAACGGGTCTTGAGGGGTTTTTTGCT | |
| | GAAAGGAGGAACTATATGCGCTCATA | |
| Transcript of Δ5′- | CCAAGAGGGGGGTGGTGATTGGCCTTTGGCTTATCAGTGTATA | 7 |
| 10AA-T2A-3′ | GATAACATTTCTCTGACCGTTTACCACACAATTAACAACAATA | |
| HAstV-nLUC | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAGCTATGGTGCATGGCAACACTCTTTCATATGCAGTTCGC | |
| | ACCCAGGACGGGATGTCGGGTGCACCAGTCTGTGACAAATATG | |
| | GTCGGGTGTTAGCAGTCCATCAAACAAACACTGGGTACACTGG | |
| | AGGTGCTGTCATAATAGACCCAGCAGACTTCCATCCAGTGAAA | |
| | GCCCCATCTCAGGTGGAATTGCTCAAAGAGGAAATAGAGCGGC | |
| | TAAAAGCTCAACTGAACTCTGCCACTGAGAACGCAACGACTGT | |
| | AGTTACACAACAACCTAGTGCTGCACTAGAACAGAAAAGTGTC | |
| | AGCGATAGTGATGTAGTTGACCTTGTCAGAACTGCAATGGAAC | |
| | GTGAGATGAAGGTGCTGCGTGATGAAATCAATGGAATACTTGC | |
| | ACCATTCCTACAAAAAAAGAAAGGTAAGACCAAGCATGGTAGG | |
| | GGTAGAGTCAGGCGTAACCTTAGAAAAGGTGTGAAACTTCTTA | |
| | CCGAGGAAGAGTATCGAGAACTCTTAGAGAAAGGTCTTGATCG | |
| | TGAGACATTCCTTGATCTCATAGACCGCATTATTGGTGAGAGG | |
| | TCTGGCTACCCTGACTATGATGATGAAGATTACTATGATGAAG | |
| | ATGATGATGGCTGGGGAATGGTTGGTGATGATGTAGAATTTGA | |
| | TTATACTGAAGTAATTAACTTTGACCAAGCAAAACCAATTCCT | |
| | GCCCCGAGAACAACCAAGCAAAAAATTTGCCCCGAACCAGAAG | |
| | TCGAATCACAACCACTTGATTTGTCCCAAAAGAAAGAAAAACA | |
| | ATCAGAATATGAACAACAAGTGGTGAAGTCTACCAAGCCTCAA | |
| | CAATTAGAACATGAACAACAAGTGGTGAAGCCTATCAAGCCTC | |
| | AGAAGAGTGAGCCTCAACCATACTCACAAACTTACGGCAAGGC | |
| | ACCAATCTGGGAATCTTACGATTTTGACTGGGATGAGGATGAT | |
| | GCCAAGTTTATTCTGCCAGCGCCACACCGGTTGACTAAGGCAG | |
| | ATGAAATAGTCCTTGGATCCAAAATCGTCAAGCTTAGAACGAT | |
| | TATTGAAACAGCCATAAAGACTCAGAATTATAGTGCATTACCT | |
| | GAAGCAGTATTTGAGCTCGACAAAGCAGCTTATGAAGCAGGTT | |
| | TGGAAGGTTTTCTCCAAAGGGTTAAATCGAAAAACAAGGCCCC | |
| | AAAAAACTACAAAGGGCCCCAGAAGACCAAGGGGCCCAAAACT | |
| | ACCACTCATTAGATGCATGGAAATTGTTGCTAGAGCCTCCGCG | |
| | GGAGCGAAGGTGCGTGCCTGCGAATTTTCCACTATTAGGCCAT | |
| | TTACCAATTAATAGACCCATCTTTGATGATAAGAAACCCAGGG | |
| | ACGATCTCCTTGGCCTACTTCCAGAACCAACCTGGCATGCTTT | |
| | CGAGGAATACGGACCAACCACATGGGGCCCACAAGCTTTTATC | |
| | AAATCTTTTGATAAATTTTTTTATGCAGAACCAATTGACTTTT | |
| | TCTCAGAATATCCACAGTTGTGTGCTTTCGCTGATTGGGCAAC | |
| | TTATCGCGAGTTTCGGTATCTAGAGGATACTAGAGTGATACAC | |
| | ATAACTGCAACTGAGAAAAACACTGATTCAACACCTGCTTATC | |
| | CTAAAATGAATTATTTTGACACTGAAGAAAATTACCTGGAAGC | |
| | ACATGGGTGGGCACCATATATTAGAGAATTCACTAGGGTCTAC | |
| | AAAGGAGACAAACCTGAAGTACTTTGGTACCTATTTCTTAAGA | |
| | AAGAGATCATTAAGGAGGAAAAAATTAGGAATTCTGATATCCG | |
| | GCAGATAGTGTGTGCCGACCCCATTTACACCAGGATAGGGGCG | |
| | TGCTTAGAAGCGCATCAGAATGCCTTAATGAAACAGCATACCG | |
| | ACACCTCAGTTGGTCAATGTGGGTGGTCACCAATGGAAGGCGG | |
| | CTTTAAAAAAACCATGCAGCGCCTGGTAAATAAAGGGAATAAA | |
| | CACTTCATTGAGTTCGACTGGACCCGCTATGATGGAACTATAC | |
| | CACCAGCACTCTTTAAACACATCAAAGAAATTAGGTGGAATTT | |
| | CATCAATAAAGACCAACGTGAAAAGTACAGACATGTACATGAA | |
| | TGGTATGTTAACAACCTCCTTAATCGCCATGTACTTCTACCAT | |
| | CTGGTGAAGTCACCTTGCAGACGCGAGGTAATCCATCTGGTCA | |
| | GTTTTCAACAACAATGGATAACAACATGGTTAACTTTTGGTTA | |
| | CAGGCTTTTGAGTTTGCTTATTTTAATGGACCAGACAGAGACC | |
| | TTTGGAAGACCTATGACACTGTAGTTTATGGAGATGACAGGCT | |
| | TTCTACAACACCTTCGGTGCCCGATGATTATGAGGAGAGAGTG | |
| | ATCACTATGTATAGAGACATCTTTGGCATGTGGGTTAAGCCTG | |
| | GGAAGGTTATCTGTAGAGACAGCATAGTTGGATTGTCCTTTTG | |
| | TGGCTTCACTGTTAATGAAAACCTTGAGCCTGTGCCAACTTCT | |
| | CCTGAAAAGTTAATGGCATCACTGTTAAAACCTTACAAAATAT | |
| | TACCTGATCTTGAATCACTCCATGGGAAACTCCTATGCTATCA | |
| | GTTGCTTGCTGCGTTCATGGCAGAGGACCACCCCTTTAAGGTG | |
| | TATGTGGAGCACTGCCTCTCGCGGACTGCAAAGCAGCTTCGTG | |
| | ACTCTGGCCTTCCAGCCAGACTCACAGAAGAGCAACTCCATCG | |
| | CATTTGGAGGGGAGGACCAAAGAAGTGTGATGGCTAGCAAGTC | |
| | CAACAAGCAAGTAACTgaaggccggggcagtctgctgacgtgc | |
| | ggcgacgtagaagaaaatcctggtcccCCTAGGATGGTCTTCA | |
| | CACTCGAAGATTTCGTTGGGGACTGGCGACAGACAGCCGGCTA | |
| | CAACCTGGACCAAGTCCTTGAACAGGGAGGTGTGTCCAGTTTG | |
| | TTTCAGAATCTCGGGGTGTCCGTAACTCCGATCCAAAGGATTG | |
| | TCCTGAGCGGTGAAAATGGGCTGAAGATCGACATCCATGTCAT | |
| | CATCCCGTATGAAGGTCTGAGCGGCGACCAAATGGGCCAGATC | |
| | GAAAAAATTTTTAAGGTGGTGTACCCTGTGGATGATCATCACT | |
| | TTAAGGTGATCCTGCACTATGGCACACTGGTAATCGACGGGGT | |
| | TACGCCGAACATGATCGACTATTTCGGACGGCCGTATGAAGGC | |
| | ATCGCCGTGTTCGACGGCAAAAAGATCACTGTAACAGGGACCC | |
| | TGTGGAACGGCAACAAAATTATCGACGAGCGCCTGATCAACCC | |
| | CGACGGCTCCCTGCTGTTCCGAGTAACCATCAACGGAGTGACC | |
| | GGCTGGCGGCTGTGCGAACGCATTCTGGCGTAATAGACGCGTg | |
| | caaagcagcaggggaaatcaatccctgcacatctggaagccgc | |
| | ggccacgccgagtagGAACGAGGGTACAGCTTCCTTCTTTTCT | |
| | GTCTCTGTTTAGATTATTTTAATCACCATTTAAAATTGATTTA | |
| | ATCAGAAGCAAAAAAAAAAAAAAAAAAAAAAAAAAAAAAAAAA | |
| | AAAAAAAAAAAGGGTCGGCATGGCATCTCCACCTCCTCGCGGT | |
| | CCGACCTGGGCATCCGAAGGAGGACGCACGTCCACTCGGATGG | |
| | CTAAGGGAGCCTGCATTCGCAGAAGCCACCCGCGCTGCTAACA | |
| | AAGCCCGAAAGGAAGCTGAGTTGGCTGCTGCCACCGCTGAGCA | |
| | ATAACTAGCATAACCCCTTGGGGCCTCTAAACGGGTCTTGAGG | |
| | GGTTTTTTGCTGAAAGGAGGAACTATATGCGCTCATA | |
| Transcript of Δ5′- | CCAAGAGGGGGGTGGTGATTGGCCTTTGGCTTATCAGTGTATA | 8 |
| HVR-IV-10AA- | GATAACATTTCTCTGACCGTTTACCACACAATTAACAACAATA | |
| T2A-3′ HAstV-nLUC | TGGCATACGGTGAGCCATACTATAGCTCTAAACCTGACAAAGA | |
| | TTTCAATTTTGGAAGCACAATGGCACGTAGGCAGATGACACCT | |
| | ACTATGGTAACAAAGCTTCCCAAATTTGTTAGGAATTCTCCAC | |
| | AAGCTTATGATTGGATCGTAAGAGGTCTGATCTTTCCCACCAT | |
| | TGGTAAAACTTATTTCCAACGAGTTGTTGTGATTACTGGTGGG | |
| | CTTGAGGATGGAACATATGGCTCATTCGCATTTGACGGTAAAG | |
| | AGTGGGTAGGGATCTACCCAATAGAGCACTTAAATCTCATGTC | |
| | ATCTTTGAAACTGATACACAAAGCCAACGCTCTTCAGGAGAGA | |
| | CTGCGTCTCTCCCAAGAAGAGAAAGCCACCCTCGCTCTGGATG | |
| | TGCAATTCCTTCAACATGAAAACGTGCGATTGAAGGAGATGAT | |
| | CCCAAAGCCAGAGCCACGGAAAATACAGATGAAGTGGATAATA | |
| | ATGGGAGCAGTGCTTACATTCTTATCTCTGATACCTGGGGGTT | |
| | ATGCGCACAGCCAGACCAACAACACCATATTTACTGACATGAT | |
| | AGCTGCCTGCAAGTACTCAACTGAGACACTAACAGAAAATCTT | |
| | GACCTTAGAATCAAGCTTGCACTAGCAAACATAACCATTAGTG | |
| | ATAAGCTAGATGCTGTGAGGCAAATTCTTAACTTTGCCTTTGT | |
| | GCCCAGAGCCCATTGGTTGAGAACTGTGTTCTATTATATCCAT | |
| | TACTATGAAATGTGGAATATTTTTATGTTTGTTCTTGCTATTG | |
| | GCACTGTCATGAGGAGCGCCCGCCCTGGTACAGACTTGGTTAC | |
| | ACTTGCAACATCCCACTTGTCTGGTTTTAGGATGGCTGTCCTA | |
| | CCCACAATTCCATTTCACACCACTATGACTTTGTGGGTTATGA | |
| | ACACACTTATGGTTTGTTATTATTTTGACAACTTGCTAGCAAT | |
| | AACATTGGCAATCTTAGCACCAATTCTTGGCATTATCTTCTTG | |
| | TGCTTCATGGAAGACTCCAACTATGTGAGCCAGATACGTGGCC | |
| | TTATTGCTACAGCAGTATTAATTGCTGGTGGGCATGCATGTTT | |
| | GACACTCACAGGCACGACCACGTCATTGTTTGTTGTCATACTA | |
| | ACCTGTAGGTTCATACGTATGGCAACTGTTTTCATTGGCACCA | |
| | GGTTCGAGATCCGTGACGCTAATGGAAAGGTTGTGGCCACTGT | |
| | ACCAACTAGGATTAAAAATGTTGCATTTGACTTTTTTCAGAAG | |
| | CTGAAGCAGTCAGGGGTGCGAGTTGGAGTCAACGAATTCGTTG | |
| | TCATAAAACCAGGTGCATTATGTGTTATAGACACCCCTGAAGG | |
| | AAAAGGGACAGGTTTCTTTTCTGGCAATGACATAGTAACAGCA | |
| | GCACATGTTGTTGGCAATAATACTTTTGTGAATGTGTGCTATG | |
| | AGGGTTTGATGTATGAAGCGAAGGTGCGGTACATGCCCGAAAA | |
| | GGATATAGCATTCTTAACTTGTCCTGGTGACCTGCATCCAACA | |
| | GCAAGATTAAAATTATCAAAGAACCCAGATTATAGTTGTGTCA | |
| | CAGTTATGGCTTATGTGAATGAGGATCTTGTGGTTTCAACCGC | |
| | AGCAgccatggtacatggcaacactctctcatatgcagttcgc | |
| | actcaagacggaatgtcaggtgcaccagtttgtgacaaatatg | |
| | gtcgagtgttagcagtccatcagactaatactgggtacactgg | |
| | aggtgctgtcataatagacccagcagacttccatccagtgaag | |
| | gccccatctcaggtggaattgctcaaagaggaaatagagcgat | |
| | taaaagcccaattaaattccaccgctgagaatccagcgactgt | |
| | tgttacacaacaacctattgctacactagagcagaaaagtgtc | |
| | agcgatagcgatgtgattgaccttgttagaactgcaatggaac | |
| | gtgagatgaaggtgctgcgcgatgaaatcaatgggatacttgc | |
| | accgttcctacaaaaaaagaaaggtaagaccaagcatggtagg | |
| | ggtagagtcagacgaaaccttaggaaaggtgtgaaacttctca | |
| | ctgaggaagaatatcgagagctcttagagaaaggtctggatcg | |
| | tgagacattcctagatctcatagaccgtattattggtgagagg | |
| | tctggctaccctgactatgatgatgaggattattatgatgaag | |
| | atgatgatggatggggtatggttggtgatgatgtagaatttga | |
| | ttataccgaagtaatcaattttgaccaagcaaaaccaactcct | |
| | gccccgagaacaagtaagccacaacaagccaacacttctcaaa | |
| | aaccccgccccgagctagaagctgaagcacaaccgcttgattt | |
| | gtctcagaagaaagagaaacaaccagaacatgagcaacaagtg | |
| | gcgaagcctaccaagatgcagaagaatgaacctcaaccatatt | |
| | cacaaacttatggcaaggcaccaatctgggaatcctatgactt | |
| | tgattgggatgaggatgacgccaagttcattottccagcgcct | |
| | caCCGGTTGACTAAGGCAGATGAAATAGTCCTTGGATCCAAAA | |
| | TCGTCAAGCTTAGAACGATTATTGAAACAGCCATAAAGACTCA | |
| | GAATTATAGTGCATTACCTGAAGCAGTATTTGAGCTCGACAAA | |
| | GCAGCTTATGAAGCAGGTTTGGAAGGTTTTCTCCAAAGGGTTA | |
| | AATCGAAAAACAAGGCCCCAAAAAACTACAAAGGGCCCCAGAA | |
| | GACCAAGGGGCCCAAAACTACCACTCATTAGATGCATGGAAAT | |
| | TGTTGCTAGAGCCTCCGCGGGAGCGAAGGTGCGTGCCTGCGAA | |
| | TTTTCCACTATTAGGCCATTTACCAATTAATAGACCCATCTTT | |
| | GATGATAAGAAACCCAGGGACGATCTCCTTGGCCTACTTCCAG | |
| | AACCAACCTGGCATGCTTTCGAGGAATACGGACCAACCACATG | |
| | GGGCCCACAAGCTTTTATCAAATCTTTTGATAAATTTTTTTAT | |
| | GCAGAACCAATTGACTTTTTCTCAGAATATCCACAGTTGTGTG | |
| | CTTTCGCTGATTGGGCAACTTATCGCGAGTTTCGGTATCTAGA | |
| | GGATACTAGAGTGATACACATAACTGCAACTGAGAAAAACACT | |
| | GATTCAACACCTGCTTATCCTAAAATGAATTATTTTGACACTG | |
| | AAGAAAATTACCTGGAAGCACATGGGTGGGCACCATATATTAG | |
| | AGAATTCACTAGGGTCTACAAAGGAGACAAACCTGAAGTACTT | |
| | TGGTACCTATTTCTTAAGAAAGAGATCATTAAGGAGGAAAAAA | |
| | TTAGGAATTCTGATATCCGGCAGATAGTGTGTGCCGACCCCAT | |
| | TTACACCAGGATAGGGGCGTGCTTAGAAGCGCATCAGAATGCC | |
| | TTAATGAAACAGCATACCGACACCTCAGTTGGTCAATGTGGGT | |
| | GGTCACCAATGGAAGGCGGCTTTAAAAAAACCATGCAGCGCCT | |
| | GGTAAATAAAGGGAATAAACACTTCATTGAGTTCGACTGGACC | |
| | CGCTATGATGGAACTATACCACCAGCACTCTTTAAACACATCA | |
| | AAGAAATTAGGTGGAATTTCATCAATAAAGACCAACGTGAAAA | |
| | GTACAGACATGTACATGAATGGTATGTTAACAACCTCCTTAAT | |
| | CGCCATGTACTTCTACCATCTGGTGAAGTCACCTTGCAGACGC | |
| | GAGGTAATCCATCTGGTCAGTTTTCAACAACAATGGATAACAA | |
| | CATGGTTAACTTTTGGTTACAGGCTTTTGAGTTTGCTTATTTT | |
| | AATGGACCAGACAGAGACCTTTGGAAGACCTATGACACTGTAG | |
| | TTTATGGAGATGACAGGCTTTCTACAACACCTTCGGTGCCCGA | |
| | TGATTATGAGGAGAGAGTGATCACTATGTATAGAGACATCTTT | |
| | GGCATGTGGGTTAAGCCTGGGAAGGTTATCTGTAGAGACAGCA | |
| | TAGTTGGATTGTCCTTTTGTGGCTTCACTGTTAATGAAAACCT | |
| | TGAGCCTGTGCCAACTTCTCCTGAAAAGTTAATGGCATCACTG | |
| | TTAAAACCTTACAAAATATTACCTGATCTTGAATCACTCCATG | |
| | GGAAACTCCTATGCTATCAGTTGCTTGCTGCGTTCATGGCAGA | |
| | GGACCACCCCTTTAAGGTGTATGTGGAGCACTGCCTCTCGCGG | |
| | ACTGCAAAGCAGCTTCGTGACTCTGGCCTTCCAGCCAGACTCA | |
| | CAGAAGAGCAACTCCATCGCATTTGGAGGGGAGGACCAAAGAA | |
| | GTGTGATGGCTAGCAAGTCCAACAAGCAAGTAACTgaaggccg | |
| | gggcagtctgctgacgtgcggcgacgtagaagaaaatcctggt | |
| | cccCCTAGGATGGTCTTCACACTCGAAGATTTCGTTGGGGACT | |
| | GGCGACAGACAGCCGGCTACAACCTGGACCAAGTCCTTGAACA | |
| | GGGAGGTGTGTCCAGTTTGTTTCAGAATCTCGGGGTGTCCGTA | |
| | ACTCCGATCCAAAGGATTGTCCTGAGCGGTGAAAATGGGCTGA | |
| | AGATCGACATCCATGTCATCATCCCGTATGAAGGTCTGAGCGG | |
| | CGACCAAATGGGCCAGATCGAAAAAATTTTTAAGGTGGTGTAC | |
| | CCTGTGGATGATCATCACTTTAAGGTGATCCTGCACTATGGCA | |
| | CACTGGTAATCGACGGGGTTACGCCGAACATGATCGACTATTT | |
| | CGGACGGCCGTATGAAGGCATCGCCGTGTTCGACGGCAAAAAG | |
| | ATCACTGTAACAGGGACCCTGTGGAACGGCAACAAAATTATCG | |
| | ACGAGCGCCTGATCAACCCCGACGGCTCCCTGCTGTTCCGAGT | |
| | AACCATCAACGGAGTGACCGGCTGGCGGCTGTGCGAACGCATT | |
| | CTGGCGTAATAGACGCGTgcaaagcagcaggggaaatcaatcc | |
| | ctgcacatctggaagccgcggccacgccgagtagGAACGAGGG | |
| | TACAGCTTCCTTCTTTTCTGTCTCTGTTTAGATTATTTTAATC | |
| | ACCATTTAAAATTGATTTAATCAGAAGCAAAAAAAAAAAAAAA | |
| | AAAAAAAAAAAAAAAAAAAAAAAAAAAAAAGGGTCGGCATGGC | |
| | ATCTCCACCTCCTCGCGGTCCGACCTGGGCATCCGAAGGAGGA | |
| | CGCACGTCCACTCGGATGGCTAAGGGAGCCTGCATTCGCAGAA | |
| | GCCACCCGCGCTGCTAACAAAGCCCGAAAGGAAGCTGAGTTGG | |
| | CTGCTGCCACCGCTGAGCAATAACTAGCATAACCCCTTGGGGC | |
| | CTCTAAACGGGTCTTGAGGGGTTTTTTGCTGAAAGGAGGAACT | |
| | ATATGCGCTCATA | |

1. A recombinant replicon nucleic acid comprising:
a. a first open reading frame comprising a subgenomic nucleic acid sequence encoding a protein of interest that can be secreted by a cell;
b. a second open reading frame comprising a nucleic acid sequence encoding a first astrovirus nonstructural protein (nsP1a) and including a hypervariable region; and
c. a third open reading frame comprising a nucleic acid sequence encoding a second astrovirus nonstructural protein (nsP1b) and including a subgenomic promoter that is situated so as to initiate transcription of the subgenomic nucleic acid sequence.

2. The recombinant replicon nucleic acid of claim 1, wherein the first open reading frame further comprises a subset of a nucleic acid sequence encoding an astrovirus structural protein (VP90).

3. The recombinant replicon nucleic acid of claim 2, wherein the subset consists of between 5 and 50 nucleotides.

4. The recombinant replicon nucleic acid of claim 3, wherein the subset consists of 30 nucleotides.

5. The recombinant replicon nucleic acid of claim 1, having the following structure: c.→b.→a.

6. The recombinant replicon nucleic acid of claim 1, further comprising an astrovirus conserved sequence element beginning within the first open reading frame and extending beyond the 3′ end of the first open reading frame.

7. The recombinant replicon nucleic acid of claim 1 having a first astrovirus genotype, wherein the hypervariable region has a second astrovirus genotype that is different from the first astrovirus genotype.

8. The recombinant replicon nucleic acid of claim 7, wherein the first astrovirus genotype is HAstV VII and the second astrovirus genotype is HAstV IV.

9. The recombinant replicon nucleic acid of claim 1, wherein the third open reading frame includes a translational upstream ribosome binding site.

10. The recombinant replicon nucleic acid of claim 1, having a 7-methylguanylate cap at its 5′ end.

11. The recombinant replicon nucleic acid of claim 1, wherein the first open reading frame further comprises a nucleic acid sequence encoding a peptide with ribosomal skipping properties.

12. The recombinant replicon nucleic acid of claim 11, wherein the peptide with ribosomal skipping properties is a 2A peptide from Thosea asigna virus capsid protein (T2A).

13. A nanoparticle comprising the recombinant replicon nucleic acid of claim 1.

14. The nanoparticle of claim 13, consisting essentially of a nanostructured lipid carrier containing the recombinant replicon nucleic acid.

15. A formulation comprising a plurality of the nanoparticle of claim 13 in a pharmaceutically acceptable carrier.

16. A composition comprising the recombinant replicon nucleic acid of claim 1, in a pharmaceutically acceptable carrier.

17. An isolated cell comprising the recombinant replicon nucleic acid of claim 1.

18. A method of treating a subject to confer an immunity on the subject, comprising administering the composition of claim 16 to the subject and thereby eliciting an immune response in the subject.

19. The method of claim 18, wherein the immune response includes CD4+ T cell activation.

20. A method of secreting a protein of interest from a cell, comprising introducing the recombinant replicon nucleic acid of claim 1 into the cell under conditions whereby the protein of interest is secreted, wherein the cell is in a cell culture thereby secreting the protein from the cell.

21. The method of claim 20, further comprising the step of harvesting the protein of interest from the cell culture.

22. A composition comprising the protein of interest produced from the method of claim 20.

23. A method of delivering a therapeutic protein of interest to a subject, comprising administering the composition of claim 22 to the subject.

24. A method of delivering a therapeutic amount of a protein of interest to a subject, comprising administering an effective amount of the isolated cell of claim 17 to a subject, wherein the protein of interest is a therapeutic protein and the cells secrete and thereby deliver a therapeutic amount of the protein of interest to the subject.
